(19) United States
(12) Patent Application Publication
(10) Pub. No.: US 2020/0291077 A1
(43) Pub. Date: Sep. 17, 2020

(54) POROUS MICROSPHERES INCLUDING MUSSEL ADHESIVE PROTEIN, AND METHOD FOR MANUFACTURING SAME

(71) Applicant: POSTECH ACADEMY-INDUSTRY FOUNDATION, Pohang-si (KR)

(72) Inventors: Hyung Joon Cha, Pohang-si (KR); Dong Pyo Kim, Pohang-si (KR); Yun Kee Jo, Nam-gu (KR); Kyoung Ik Min, Daejeon (KR)

(21) Appl. No.: 16/497,934

(22) PCT Filed: Mar. 27, 2018
(86) PCT No.: PCT/KR2018/003585

§ 371 Date: Sep. 26, 2019

(30) Foreign Application Priority Data
Mar. 31, 2017 (KR) 10-2017-0041584
Sep. 19, 2017 (KR) 10-2017-0120506

(51) Int. Cl. C07K 14/435; C12M 1/12; A61K 47/64; A61K 9/00

(52) U.S. Cl. CPC C07K 14/43504; A61K 9/0019; A61K 47/64; C12M 25/14

(57) ABSTRACT

The present invention relates to a porous microsphere comprising a mussel adhesive protein and a method of preparing the same. The porous microsphere comprising the mussel adhesive protein according to the present invention is capable of minimally invasive bio-injection through syringes to efficiently deliver therapeutic stem cells to the sites of tissue defects as cell carriers. Further, the present invention may be widely applied to scaffolds for tissue engineering, drug carriers, or the like, which may be suitably applied to the size of the defected site of tissue.

### TECHNICAL FIELD

The present invention relates to a porous microsphere comprising a mussel adhesive protein and method for preparing the same.

### BACKGROUND ART

Stem cells have a differentiation ability to develop into various tissues. In the tissue engineering and regenerative medicine field, studies have been actively conducted to transplant stem cells, thereby restoring human tissues or organs and to treat diseases. In particular, direct injection of stem cells into biological tissues can be applied in a minimally invasive manner so that it has been attracting attention in the clinical field. However, there are the limitations of low cell delivery efficiency such as damage or death of cells easily caused during the injection.

Microspheres (MS) are three-dimensional structures with a diameter of 1 μm to 1000 μm and have a high surface area-volume ratio, allowing efficient cell loading. Therefore, their applicability is increasing as an injection-type cell carrier capable of filling tissue defects having various shapes and sizes in a minimally invasive manner in various fields such as orthopedic and oral-facial surgery. Meanwhile, the microspheres can be used for the purpose of delivering drugs to the desired site by loading drugs therein. Because such microspheres must be introduced into the living body, their biocompatibility and biodegradability are required, and the cell attachment and growth therein should be easy based on an appropriate level of mechanical properties.

Currently, microspheres are mainly based on synthetic polymers such as polylactide glycolide copolymer (PLGA), polyhydroethyl methacrylate (Poly-HEMA), polystyrene, and polyacrylamide (PAA). Such synthetic polymer-based microspheres are advantageous in that they are easy to control mechanical properties and biodegradation rate by easily controlling molecular structure and molecular weight. However, due to their low biocompatibility and lack of functionality for direct interaction with cells, there is a problem that ultimately results in loss of cell viability or difficulty in controlling cell differentiation capacity. On the other hand, natural polymers such as collagen and chitosan have similar properties to extracellular substrates and have excellent biocompatibility, which has been suggested as an alternative for in vivo application. However, there is a limit of low retention in the cell delivery environment due to their low mechanical properties.

Therefore, in order to overcome these problems, it is necessary to develop ideal biomaterial-based microspheres that can minimize side effects in the living body while providing an environment in which cells can be efficiently attached and grown based on an appropriate level of mechanical properties.

Meanwhile, mussels, which are marine organisms, generate and secrete adhesive proteins, and thus they can be firmly attached to wet solid surfaces such as rocks in the sea so that they are not affected by shock of the waves or buoyancy effects of seawater. The mussel adhesive protein is known as a strong natural adhesive, which has high biocompatibility and biodegradability as well as high tensile strength and flexibility compared to chemical synthetic adhesives. In addition, mussel adhesive proteins have the ability to adhere to a variety of surfaces, including plastics, glass, metals, Teflon and biomaterials and adhesion on wet surfaces is also possible in minutes, which remains an unfinished task in the development of chemical adhesives.

In particular, it is known that the 3,4-dihydroxyphenylalanine (DOPA) residues present in the mussel adhesive protein play an important role in surface adhesion and bind to surrounding DOPA residues and amino acids while oxidizing to DOPA-quinone to enable excellent mechanical properties of mussel byssus as a crosslinking mediator causing crosslinking.

However, to date, there has been no attempts to make microspheres or to apply them as cell carriers using oxidization of DOPA contained in mussel adhesive proteins.

### DISCLOSURE

### Technical Problem

In the previous study, the present inventors developed new form of mussel adhesive protein fp-151, where decapeptide that repeats about 80 times in Mefp-1 is linked 6 times consecutively and fused at both ends of Mgfp-5, and identified that the mussel adhesive protein could be mass-produced in E. coli, and the purification process is very simple, and thus, the industrial applicability is very high (International Patent Publication No. WO2006/107183 or WO2005/092920).

Based on this mass production technology, the present inventors convert tyrosine residues of mussel adhesive proteins mass-produced in E. coli system into DOPA with high efficiency and prepares mussel adhesive protein-based porous microspheres using simple microfluidic process (microfluidic device). The porous microspheres thus prepared were confirmed to exhibit excellent efficacy as cell and drug carriers, thereby completing the present invention.

Accordingly, an object of the present invention is to provide a porous microsphere comprising a mussel adhesive protein and a method of preparing the same.

In addition, an object of the present invention is to provide a composition for forming a porous microsphere, in which the composition comprises a mussel adhesive protein.

In addition, an object of the present invention is to provide a cell carrier, a scaffold for tissue engineering, and a drug carrier using the porous microsphere.

In addition, an object of the present invention is to provide a method for cell or drug delivery in which the method includes administering the porous microsphere to a subject.

### Technical Solution

In order to achieve the objects, the present invention provides a porous microsphere comprising a mussel adhesive protein.

Further, the present invention provides a composition for forming a porous microsphere in which the composition comprises a mussel adhesive protein.

Further, the present invention provides a cell carrier comprising the porous microsphere.

Further, the present invention provides a scaffold for tissue engineering comprising the porous microsphere.

Further, the present invention provides a drug carrier comprising the porous microsphere.

Further, the present invention provides a method for preparing a porous microsphere, in which the method comprises: 1) forming a water-in-oil emulsion comprising a mussel adhesive protein, an oxidizing agent and a porogen inside a microfluidic channel; 2) crosslinking the water-in-oil emulsion through oxidation of DOPA residues contained in the mussel adhesive protein; and 3) washing the emulsion particle obtained after crosslinking and forming a pore.

Further, the present invention provides a method for cell delivery, in which the method comprises administering the porous microsphere to a subject.

Further, the present invention provides a method for drug delivery, in which the method comprises administering the porous microsphere to a subject.

### Advantageous Effects

The porous microsphere comprising the mussel adhesive protein according to the present invention is capable of minimally invasive bio-injection through syringes to efficiently deliver therapeutic stem cells to the sites of tissue defects as cell carriers. Further, the present invention may be widely applied to scaffolds for tissue engineering, drug carriers, or the like, which may be suitably applied to the size of the defected site of tissue.

### DESCRIPTION OF DRAWINGS

FIG. 1 illustrates a schematic diagram of a method for preparing the cell carrier comprising the mussel adhesive protein-based porous microsphere of the present invention.

FIG. 2 shows the results of analyzing the conversion rate of the mussel adhesive protein fp-151 tyrosine residues to the DOPA residues using an amino acid analyzer.

FIG. 3 is an image of showing a process of forming a water-in-oil emulsion comprising mussel adhesive protein, an oxidizing agent and a porogen in a microfluidic channel composed of a fluoroethylene propylene-polyimide (FEP-PI).

FIG. 4 is an image over time of showing a series of states in which the emulsion formed through the microfluidic channel is condensed by cross-linking through the oxidation reaction of the DOPA residues contained in the mussel adhesive protein (a: an image immediately after emulsion formation, b: an image of 12 hours after emulsion formation, and c: an image of 24 hours after emulsion formation).

FIG. 5 is a graph illustrating the size of each emulsion particle in the emulsion condensation process of FIG. 4.

FIG. 6 shows the results observed using a scanning electron microscope (SEM) after 24 hours of the emulsion condensation.

FIG. 7 shows the result of observing the process of washing the emulsion crosslinked through the condensation process sequentially with ethanol, acetone and ethanol-hydrochloric acid mixture, swelling and generating pores to form a porous microsphere in the process (a, b: FITC-conjugated microspheres and rhodamine-conjugated microspheres observed using a fluorescent microscope, c: a structure of porous microsphere observed using SEM).

FIG. 8 shows the result of confirming that DOPA is converted into DOPA-quinone (1240 cm⁻¹) by an oxidizing agent through FT-IR analysis of the surface of the porous microsphere (MAP-PMS: fp-151 forming a porous microsphere, MAP+NaIO₄: fp-151 treated with NaIO₄, and MAP: modified fp-151).

FIG. 9 shows the results of confirming the charge on the surface of the porous microsphere.

FIG. 10 shows the results of confirming the pore distribution of the sectional surface of the porous microsphere by SEM.

FIG. 11 shows the results of confirming the pore distribution of the sectional surface of the porous microsphere through micro-computed tomography (micro-CT).

FIG. 12 is an image showing the introduction of the porous microsphere into the syringe for the application of the porous microsphere (a: porous microsphere powders, b: porous microspheres mixed in PBS solution, c: porous microspheres introduced in a 21-gauge syringe).

FIG. 13 is a photograph taken in time order of the appearance of the porous microsphere discharged through the syringe.

FIG. 14 shows the structure prepared in the form of a cylinder using a mold for the use of porous microspheres as a scaffold for filling tissue defect sites.

FIG. 15 shows the results confirming the excellent adhesion on the skin surface of animal tissues of the porous microsphere (MAP-PMS) of the present invention compared to the control (alginate microspheres, Alginate MS).

FIG. 16 shows cell adhesion efficiency of porous microspheres on mesenchymal stem cells (rMSCs), periodontal ligament stem cells (hPLDCs) and human umbilical vein endothelial cells (HUVECs).

FIG. 17 is a graph illustrating the growth pattern for each cell according to the culture date of the porous microsphere.

FIG. 18 shows the results of observing adhesion and growth of porous microspheres on mesenchymal stem cells (a: methylene blue staining results after 1 day from cell attachment, b: SEM observation results of microspheres after 1 day from cell attachment, and c to f: fluorescence staining results after 0, 1, 4 and 7 days immediately after cell attachment).

FIG. 19 shows the results of confirming successful bio-injection of microspheres by minimally invasive injection (a: mouse observation results on the day of injection, b: mouse observation results after 28 days from the injection, and c and d: fluorescence assay results on injection day and after 28 days therefrom).

FIG. 20 shows a schematic diagram of the minimally invasive bio-injection through a syringe with porous microspheres comprising mussel adhesive proteins according to the present invention.

### MODES OF THE INVENTION

The present invention provides a porous microsphere comprising mussel adhesive protein.

In the present invention, “microsphere” means a three-dimensional structure having a diameter of 1 μm to 1000 μm. The diameter of the microspheres may be preferably 1 μm to 500 μm, more preferably 1 μm to 250 μm. The microspheres can be used for two-dimensional to three-dimensional cell culture by attaching the surrounding cells present in the medium to the surface.

The “porous microsphere” of the present invention is a porous particle having uniform pores and may include mussel adhesive proteins to be characterized by excellent biocompatibility and adhesion.

In the present invention, “mussel adhesive protein” is an adhesive protein derived from mussels, preferably an adhesive protein derived from Mytilus edulis, Mytilus galloprovincialis, Mytilus coruscus or variants thereof, but are not limited thereto.

For example, the mussel adhesive protein of the present invention may include Mefp (Mytilus edulis foot protein)-1, Mefp-2, Mefp-3, Mefp-4, Mefp-5, Mgfp (Mytilus galloprovincialis foot)-1, Mgfp-2, Mgfp-3, Mgfp-4, Mgfp-5, Mcfp (Mytilus coruscus foot protein)-1, Mcfp-2, Mcfp-3, Mcfp-4, and Mcfp-5 or variants thereof and preferably a protein selected from the group consisting of fp (foot protein)-1 (SEQ ID NO: 1), fp-2 (SEQ ID NO: 5), fp-3 (SEQ ID NO: 6), fp-4 (SEQ ID NO: 7), fp-5 (SEQ ID NO: 8), and fp-6 (SEQ ID NO: 9), or a fusion protein to which one or more proteins selected from the group are linked, or variants thereof, but are not limited thereto.

In addition, the mussel adhesive protein of the present invention may include all mussel adhesive proteins described in WO2006/107183 or WO2005/092920. Preferably, the mussel adhesive protein may include fusion proteins such as fp-151 (SEQ ID NO: 10), fp-131 (SEQ ID NO: 12), fp-353 (SEQ ID NO: 13), fp-153 (SEQ ID NO: 14), fp-351 (SEQ ID NO: 15), but are not limited thereto.

In addition, the mussel adhesive protein of the present invention may include a polypeptide where decapeptide (SEQ ID NO: 2) that repeats about 80 times in fp-1 (SEQ ID NO: 1) is linked 1 to 12 times or more, preferably a fp-1 variant polypeptide (SEQ ID NO: 3) where decapeptide of SEQ ID NO: 2 is linked 12 times consecutively, but is not limited thereto.

Also, under the condition that the mussel adhesive protein maintains adhesion of the mussel adhesive protein, the mussel adhesive protein of the present invention may include an additional sequence to the carboxyl terminus or the amino terminus of the mussel adhesive protein or substitute some amino acids with other amino acids. Preferably, the mussel adhesive protein may have a polypeptide of 3 to 25 amino acids containing Arg-Gly-Asp (RGD) linked to the carboxyl terminus or the amino terminus of the mussel adhesive protein, but is not limited thereto.

The 3 to 25 amino acids containing RGD is preferably, but not limited to, at least one selected from the group consisting of RGD (Arg Gly Asp, SEQ ID NO: 16). RGDS (Arg Gly Asp Ser, SEQ ID NO: 17), RGDC (Arg Gly Asp Cys. SEQ ID NO: 18). RGDV (Arg Gly Asp Val, SEQ ID NO: 19), RGDSPASSKP (Arg Gly Asp Ser Pro Ala Ser Ser Lys Pro, SEQ ID NO: 20), GRGDS (Gly Arg Gly Asp Ser, SEQ ID NO: 21), GRGDTP (Gly Arg Gly Asp Thr Pro, SEQ ID NO: 22), GRGDSP (Gly Arg Gly Asp Ser Pro, SEQ ID NO: 23), GRGDSPC (Gly Arg Gly Asp Ser Pro Cys, SEQ ID NO: 24) and YRGDS (Tyr Arg Gly Asp Ser, SEQ ID NO: 25).

Examples of the variant of the mussel adhesive protein to which a polypeptide of 3 to 25 amino acids containing RGD linked to the carboxyl terminus or the amino terminal of the mussel adhesive protein may be a fp-1 variant-RGD polypeptide including the amino acid sequence represented by SEQ ID NO: 4 or a fp-151-RGD polypeptide including the amino acid sequence represented by SEQ ID NO: 11, but is not limited thereto.

In the present invention, the mussel adhesive protein may be a protein consisting of the amino acid sequence represented by SEQ ID NO: 10.

Further, in the present invention, 10% to 100% of the total tyrosine residues in the mussel adhesive protein may be converted into DOPA. Tyrosine forms about 20% to 30% of the total amino acid sequences of almost all of the mussel adhesive proteins. Tyrosine in a natural mussel adhesive protein is converted into a form of DOPA by adding an —OH group through a hydration process. However, for a mussel adhesive protein produced in Escherichia coli, tyrosine residues are not converted, and thus, it is preferable to conduct a modification reaction of converting tyrosine residues into DOPA by a separate enzyme and chemical process. For a method for modifying tyrosine residues included in the mussel adhesive protein into DOPA, any known methods in the art may be used without specific limitation. As a preferable example, tyrosine residues may be modified to DOPA residues using tyrosinase. According to an embodiment of the present invention, a mussel adhesive protein satisfying the DOPA conversion rate is produced through an in vitro enzymatic reaction using mushroom tyrosinase.

In the present invention, the porous microsphere may be formed from a water-in-oil emulsion. The water-in-oil emulsion may be condensed through cross-linking by the oxidation reaction of the DOPA residues contained in mussel adhesive proteins.

Further, in the present invention, the porous microsphere may be characterized as having biocompatibility, and the porous microsphere may be characterized by loading or attaching to the bioactive substance.

The microspheres of the present invention can be easily loaded with or attached to various bioactive substances involved in the action of promoting the cell growth and differentiation through the interaction with cells or tissues of the human body as well as aiding the tissue regeneration and recovery.

In the present invention, “bioactive substance” is a generic term for various biomolecules, which includes a cell, a protein, a nucleic acid, a sugar, an enzyme, and the like. The bioactive substance may be a cell, a protein, a polypeptide, a polysaccharide, a monosaccharide, an oligosaccharide, a fatty acid, a nucleic acid, and preferably a cell. The “cell” may be any cell including a prokaryotic and eukaryotic cell. Examples of cell include mesenchymal stem cells, adipose stem cells, osteoblasts, periodontal ligament cells, vascular endothelial cells, fibroblasts, hepatocytes, neurons, cancer cells, immune cells including B cells, white blood cells and embryonic cells, etc. In addition, the bioactive substance includes plasmid nucleic acid as a nucleic acid substance, hyaluronic acid, heparin sulfate, chondroitin sulfate as sugar substances, alginate, and hormonal protein as a protein substance, but is not limited thereto.

Further, the present invention provides a composition for forming porous microspheres in which the composition comprises a mussel adhesive protein.

The present invention provides a composition for forming porous microspheres in which the composition may further comprise an oxidizing agent and porogen.

In the present invention, the “oxidizing agent” may be NaIO₄, NaIO₃, VOSO₄, Na₃VO₄, Na₂Cr₂O₇, Mn(OAc)₃, MnO₂, KMnO₄, Na₂S₂O₈, H₂O₂, Na₂S₂O₄, BHP (tert-butyl hydroperoxide), DTT (dithiothreitol), etc. Preferably, NaIO₄ can be used to oxidize DOPA residues.

In the present invention, the “porogen” may be a gas-forming carbonate such as ammonium bicarbonate, sodium bicarbonate and calcium carbonate, preferably ammonium bicarbonate, but is not limited thereto.

Further, the present invention provides a cell carrier comprising a porous microsphere of the present invention.

The cell carrier of the present invention may be used for the biological tissue regeneration and recovery by injecting in a minimally invasive manner with a syringe. The term “biological tissue” herein is not particularly limited and includes, for example, skin, bones, ligaments, nerves, blood vessels, muscles, eyes, brain, lungs, liver, heart, bladder, kidneys, stomach, small intestine, rectum, etc.

Therefore, the porous microsphere of the present invention can be applied in a minimally invasive manner with a syringe to a site that requires tissue regeneration and recovery, thereby delivering cells as well as filling tissue defects, or delivering drugs.

In a specific embodiment of the present invention, the possibility of the porous microsphere comprising a mussel adhesive protein as a cell carrier has been confirmed. More specifically, each medium containing mesenchymal stem cells, periodontal ligament stem cells and human umbilical vein endothelial cell is mixed and adhered to the porous microsphere, and cultured on the medium for six hours. Thereafter, when measuring the cell adhesion, it is confirmed that they show excellent cell loading efficiency.

In addition, the present invention provides a scaffold for tissue engineering comprising the porous microspheres of the present invention.

Tissue engineering technology refers to culturing cells isolated from a patient's tissue on a scaffold or forming a scaffold together with a scaffolding element to prepare a scaffold complex including the cells, and then transplanting the cells into the human body. Tissue engineering technology can be applied to the regeneration of almost all organs of the human body such as artificial skin, artificial cartilage, artificial bone, artificial blood vessels, and artificial muscle. It is important to form a scaffold that is harmless to cells and has properties similar to those of actual biological tissues. The porous microsphere of the present invention can be applied in the desired volume and shape to effectively fill the defects of irregularly structured tissues, as well as to provide biological tissue-like scaffold in order to optimize the biological tissue and organ regeneration in tissue engineering technology. In addition, the scaffold of the present invention may be used to easily implement artificial extracellular matrix and can be used as a medical material such as cosmetics, wound-covering material and dental matrix.

Further, the present invention provides a drug carrier comprising the porous microspheres of the present invention. The porous microsphere according to the present invention can be used as an artificial extracellular matrix of an effective scaffold for drug delivery. The drug is not particularly limited and may include chemicals, small molecules, peptide, protein medicines, nucleic acids, viruses, antibacterial agents, anticancer agents, anti-inflammatory agents, etc.

The “small molecule” may be a contrast agent (for example, a T1 contrast agent, a T2 contrast agent such as a superparamagnetic substance, a radioisotope, etc.), a fluorescent marker, a dyeing agent, etc, but is not limited thereto.

The “peptide or protein medicine” includes hormones, hormone analogs, enzymes, enzyme inhibitors, signaling proteins or parts thereof, antibodies or parts thereof, single-chain antibodies, binding proteins or binding domains thereof, antigens, adhesion proteins, structural proteins, regulatory proteins, toxin proteins, cytokines, transcriptional regulators, blood clotting factors, vaccines, etc, but is not limited thereto. More specifically, it includes fibroblast growth factor (FGF), vascular endothelial growth factor (VEGF), transforming growth factor (TGF), bone morphogenetic protein (BMP), human growth hormone (hGH), pig growth hormone (pGH), granulocyte-colony stimulating factor (G-CSF), erythropoietin (EPO), macrophage colony stimulating factor (M-CSF), tumor necrosis factor (TNF), epidermal growth factor (EGF), platelet-induced growth factor (PDGF), interferons, interleukins, calcitonin, nerve growth factor (NGF), growth hormone releasing factor, angiotensin, luteinizing hormone releasing hormone (LHRH), luteinizing hormone releasing hormone agonist (LHRH agonist), insulin, thyroid-stimulating hormone-releasing hormone (TRH), angiostatin, endostatin, somatostatin, glucagon, endorphins, bacitracin, mergain, colistin, single antibodies, vaccines or mixture thereof, but it is not limited thereto.

The “nucleic acid” may be RNA. DNA or cDNA, and the sequence of nucleic acids may be coding site sequences or non-coding site sequences (for example, antisense oligonucleotides or siRNAs).

The “virus” can be whole virus or viral core including a nucleic acid of the virus (i.e., a nucleic acid of a virus packaged without the envelope of the virus). Examples of viruses and viral cores that may be transported include papilloma virus, adenovirus, baculovirus, retrovirus core, semliki virus core, etc, but are not limited thereto.

The “antibacterial agent” may be penicillin-based agents such as penicillin, methicillin, oxacillin, nafcillin, ampicillin, carboxypenicillin, amoxicillin, and piperacillin: cephalosporins-based agents such as cephalosporin, cephazoline, ceftazidime, cefoperazone, cefotaxime, ceftizoxime, ceftriaxone, and ceftazidime; other beta-lactam-based agents such as carbapenem, meropenem, sulbactam, clavulanate, and tazobactam; aminoglycoside-based agents such as streptomycin, neomycin, gentamycin, tobramycin, amikacin, sisomycin, astromycin, iseparmycin, arbekacin; macrolide-based agents such as erythromycin and clarithromycin; tetracycline-based agents such as tetracycline, metacycline, minocycline, tigecycline and doxycycline: glycopeptide-based agent such as vancomycin and teicoplanin; lincomycin-based agent such as lincomycin and clindamycin; quinolone-based agents such as nalidixic acid, oxolinic acid, fluoroquinolone, ciprofloxacin, norfloxacin, levofloxacin; rifamycin, chloramphenicol, polymycin, trimethoprim, streptogramin, oxazolidinone, bacitracin and mixtures thereof, but is not limited thereto.

The anticancer agent may be paclitaxel, taxotere, adriamvcin, endostatin, angiostatin, mitomycin, bleomycin, cisplatin, carboplatin, doxorubicin, daunorubicin, idarubicin, 5-fluorouracil, methotrexate, actinomycin-D and mixtures thereof, but is not limited thereto.

The anti-inflammatory agents are acetaminophen, aspirin, ibuprofen, diclofenac, indomethacin, piroxicam, fenoprofen, flurbiprofen, ketoprofen, naproxen, suprofen, loxoprofen, cinnoxicam, tenoxicam, and mixtures thereof, but is not limited thereto.

Further, the present invention provides a method for preparing a porous microsphere, the method comprising: 1) forming a water-in-oil emulsion comprising a mussel adhesive protein, an oxidizing agent and a porogen inside a microfluidic channel, 2) crosslinking the water-in-oil emulsion through oxidation of DOPA residues contained in the mussel adhesive protein: and 3) washing the emulsion particle obtained after crosslinking and forming pores.

In step 1), a surfactant-dissolved oil phase and an aqueous phase in which a mussel adhesive protein containing DOPA, an oxidizing agent and porogen are dissolved are introduced into a microfluidic channel at a constant speed using a syringe pump, thereby continuously generating a water-in-oil emulsion in a uniform size. At this time, the emulsion can be formed by mixing the mussel adhesive protein, the oxidizing agent and the porogen at various concentrations.

The oil phase may be one or more selected from the group consisting of vegetable oil, mineral oil, silicone oil and synthetic oil, and may preferably be fluorinated oil.

The surfactant is generally a surfactant that can stabilize the water-in-oil emulsion, and may include at least one selected from the group consisting of fluorosurfactant, Tween®-20, Tween®-80, Pluronic® F108, Span-80, sodium dodecyl sulfate (SDS), sodium lauryl sulfate (SLS) and may preferably be fluorosurfactant.

In step 1), the microfluidic channel for forming an emulsion may preferably be a Ψ-type microchannel composed of a hydrophobic surface of fluoroethylene propylene-polyimide (FEP-PI), but not limited thereto.

In step 1), the oxidizing agent is an oxidizing agent for oxidizing the DOPA included in the mussel adhesive protein, and NaIO₄ is preferably used, but is not limited thereto. Preferably, a NaIO₄ solution may be introduced into the aqueous phase so that the ratio of DOPA:IO₄⁻ molecules is 2:1 to 4:1 in the solution in which the mussel adhesive protein is dissolved at 10 wt % to 20 wt %. More preferably, a NaIO₄ solution may be introduced so that the ratio of DOPA:IO₄⁻ molecules is 2:1.

In step 1), the porogen may be a gas-forming carbonate such as ammonium bicarbonate, sodium bicarbonate and calcium carbonate, and can be introduced into the water phase by adding 2 wt % to 5 wt % ammonium bicarbonate solution in the solution in which the mussel adhesive protein is dissolved at 10 wt % to 20 wt %.

Step 2) is a process of crosslinking the DOPA residues through oxidation reaction, wherein the DOPA residues are contained in the mussel adhesive protein in the emulsion obtained in step 1). When DOPA is oxidized, it is converted into a form of DOPA-quinone, thereby causing DOPA-DOPA bonding or allowing the DOPA-quinone to bind to the surrounding amine group or thiol group.

In step 3), in the process of washing the crosslinked emulsion particles of step 2), the oil and the surfactant covering the emulsion particles are removed and the particles are swollen as the lattice of the protein molecules constituting the microspheres is filled with water. At the same time, porous microspheres are formed as the pores are created by gas generation by porogen.

In step 3), one or more selected from the group consisting of ethanol, methanol, isopropyl alcohol, acetone, hydrochloric acid and sodium hydroxide may be used to wash the emulsion particles, but is not limited thereto.

In the porous microsphere, the diameter of the generated pores may be 10 μm to 50 μm, preferably 20 μm to 40 μm, and more preferably 20 μm to 30 μm. In addition, the porosity of such porous microsphere may be 50% to 95%, preferably 70% to 95%, more preferably 90% to 95%.

The porous microsphere of the present invention produced by the preparation method has a pore structure to attach the cells inside the pores, thereby enabling the microsphere to transfer more cells, to effectively protect the cells from the external environment through inner microenvironment of the separated pores, and to enable effective fluid circulation, oxygen and nutrient supply. Thus, the porous microsphere is applicable for various biomedical fields such as cell carriers, tissue engineering scaffolds, drug carriers, etc.

Further, the present invention provides the method for cell delivery, in which the method includes administering the porous microsphere to a subject.

Further, the present invention provides the method for drug delivery, in which the method includes administering the porous microsphere to a subject.

Hereinafter, the present invention is described in detail with examples. However, the following examples are merely to illustrate the present invention, the present invention is not limited by the following examples.

### Example 1. Preparation of Mussel Adhesive Protein-Based Porous Microsphere

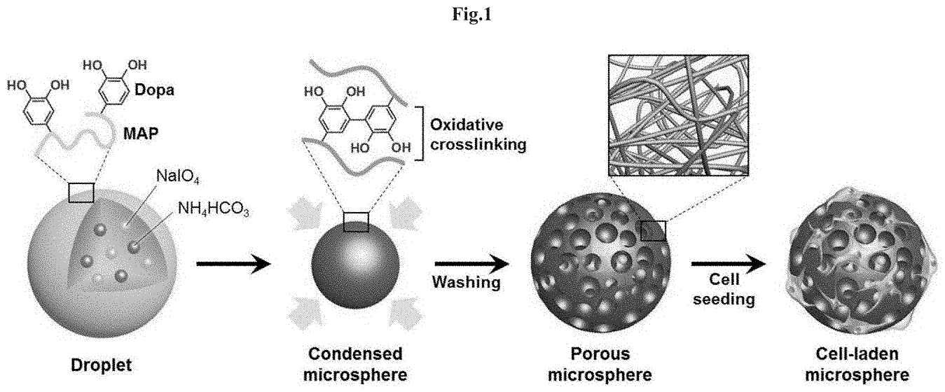

In order to prepare porous microspheres based on mussel adhesive protein, the following experiments were carried out sequentially. A scheme of the overall experiment for preparing the porous microsphere of the present invention is shown in FIG. 1.

1-1. Preparation of Mussel Adhesive Protein fp-151

For decapeptide which repeats about 80 times in natural mussel adhesive protein fp-1 and consists of 10 amino acids to be expressed in E. coli, the mussel adhesive protein fp-1 variant composed of 6 decapeptides was prepared. Mgfp-5 gene (Genbank No. AAS00463 or AY521220) was inserted between two fp-1 variants to allow successful expression in E. coli. Subsequently, the mussel adhesive protein fp-151 was produced through purification process using acetic acid (D. S. Hwang et. al, Biomaterials 28, 3560-3568, 2007).

Specifically, in the amino acid sequence of fp-1 (Genbank No. Q27409 or S23760), a fp-1 variant (hereinafter, referred to as 6xAKPSYPPTYK) represented by SEQ ID NO: 3, in which the peptide consisting of AKPSYPPTYK represented by SEQ ID NO: 2 is repeatedly linked 6 times, was prepared. The mussel adhesive protein fp-151 was prepared by combining 6xAKPSYPPTYK at the N-terminus of fp-5 and 6xAKPSYPPTYK at the C-terminus of fp-5. It was successfully expressed in E. coli and produced through purification separation using acetic acid. Specific preparation of the mussel adhesive protein is the same as that shown in WO2006/107183 or WO2005/092920, which is incorporated by reference in the present invention as a whole. The prepared fp-151 is shown in SEQ ID NO: 10.

1-2. DOPA Modification Reaction

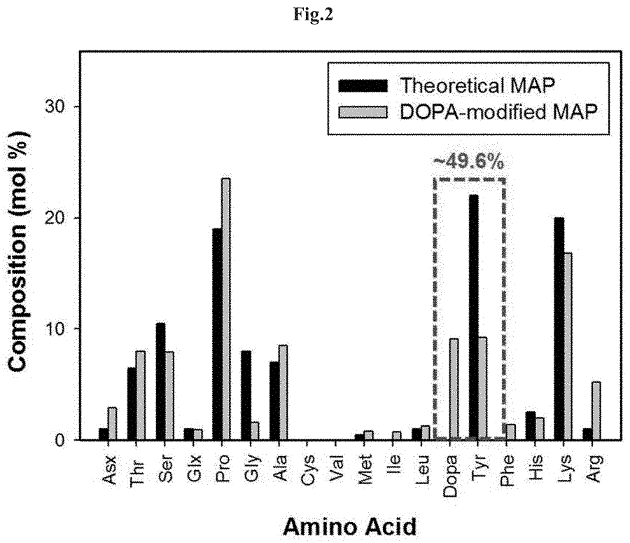

In order to convert the tyrosine residues constituting mussel adhesive protein fp-151 prepared as described above into DOPA, a modification reaction was carried out in vitro using a tyrosinase enzyme (mushroom tyrosinase). Specifically, 150 mg of lyophilized mussel adhesive protein fp-151 and 5 mg of tyrosinase were dissolved in 100 ml of a buffer solution (0.1 M sodium phosphate, 20 mM boric acid, 25 mM ascorbic acid, pH 6.8) for the modification reaction and reacted for 1 hour. Thereafter, three times dialysis for more than at least 4 hours were performed using 3 L of 25% acetic acid solution, followed by lyophilization. In order to analyze the modification efficiency of the mussel adhesive protein fp-151, amino acid composition analysis was performed, and the results are shown in FIG. 2.

As shown in FIG. 2, it was confirmed that about 49.6% of the total tyrosine residues were converted into DOPA through the peak intensities of the DOPA and tyrosine residues.

1-3. Formation of Water-in-Oil Emulsions in Microfluidic Channels

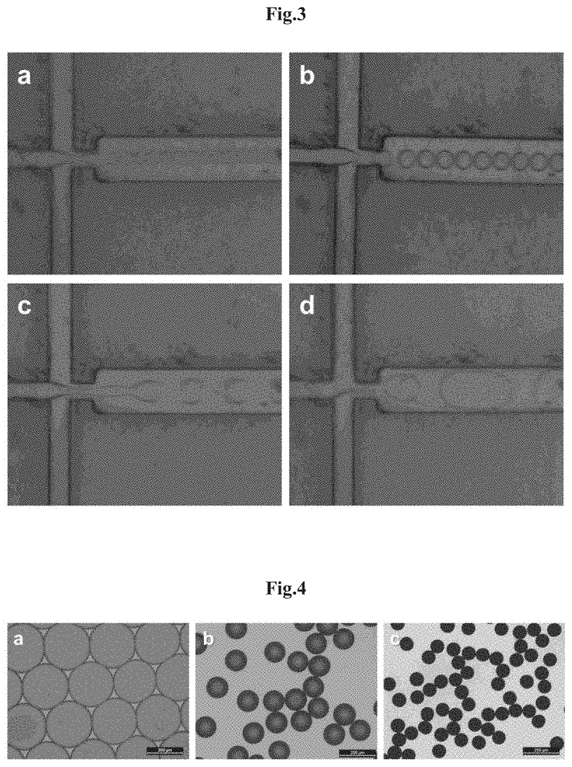

In order to prepare microspheres using the mussel adhesive protein fp-151 produced in Example 1-1, a microfluidic channel composed of fluoroethylene propylene-polyimide (FEP-PI) component and crosslinking reaction based DOPA-DOPA interaction via oxidation of NaIO₄ were used. Specifically, the modified fp-151 was dissolved in distilled water at a concentration of 10 wt % to 20 wt %, and the NaIO₄ solution was added so that the ratio of DOPA:IO₄⁻ becomes 2:1. Then, the solution was introduced with 2 wt % to 5 wt % ammonium bicarbonate as dispersed water phase, and with 1 wt % to 2 wt % mixture solution of 008-fluorosurfactant and FC-40 as continuous oil phase. Both fluids were introduced into the microfluidic channel at a constant rate of 10 μL/min and 15 μL/min using a syringe pump, respectively, and the results are shown in FIG. 3.

As shown in FIG. 3, it was confirmed that a uniform water-in-oil emulsion having a diameter of about 260 μm was formed continuously. The water-in-oil emulsion comprises a mussel adhesive protein, an oxidizing agent and a porogen.

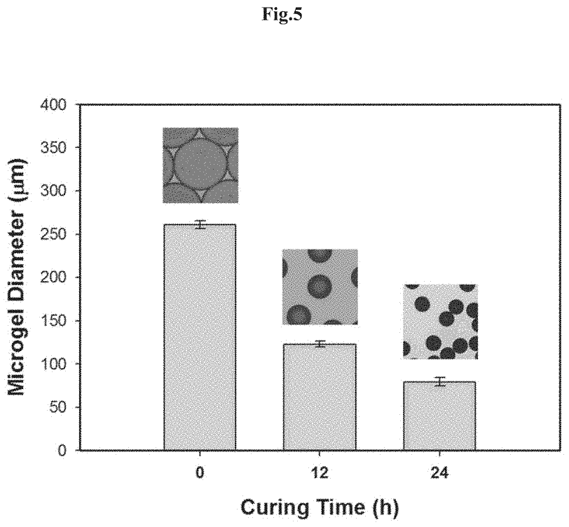

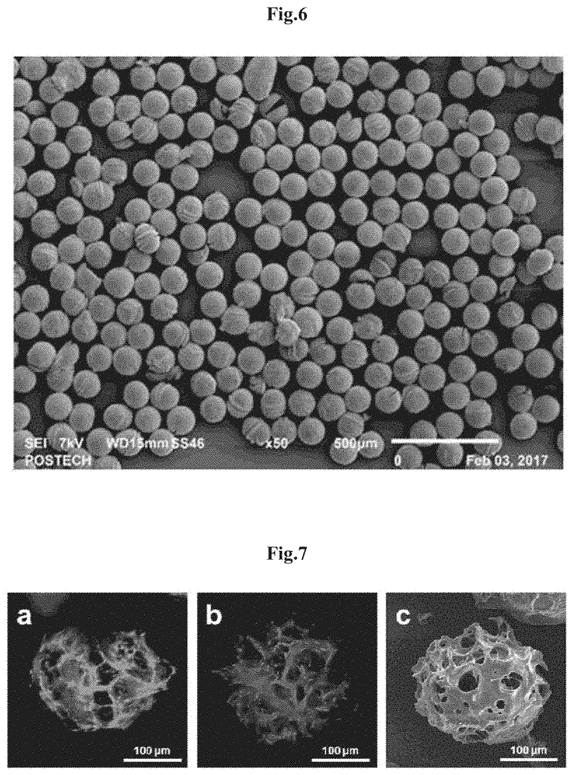

Thereafter, the emulsion prepared as described above was stored at 37° C. for 24 hours in order to crosslink through the oxidation reaction of the DOPA residues contained in the mussel adhesive protein fp-151. The results are shown in FIG. 4. FIG. 4A shows the state immediately after the emulsion is formed, FIG. 4B shows the state 12 hours after the emulsion formation, and FIG. 4C shows the state 24 hours after the emulsion formation. In addition, a graph illustrating the size of each emulsion particle in the emulsion condensation process is shown in FIG. 5. The emulsion after condensation for 24 hours observed using a scanning electron microscope (SEM) is shown in FIG. 6.

As shown in FIGS. 4 to 6, it was confirmed that as the emulsion is crosslinked through oxidation of the DOPA residues, the size of the emulsion becomes smaller, and the color becomes dark reddish brown, thereby gel-type condensed emulsion particles are obtained. It was confirmed that the condensed particles formed after the crosslinking reaction for 24 hours had a diameter of about 80 μm.

1-4. Formation of Porous Microspheres Using Mussel Adhesive Protein fp-151

The above crosslinked emulsion particles were washed sequentially using ethanol, acetone and ethanol-hydrochloric acid mixture. In this process, the oil and the surfactant covering the emulsion particles were removed, and water was filled in the lattice of the protein molecule to swell. At the same time, pores were created by gas generation due to porogen, thereby forming porous microspheres. In order to clearly observe the porous structure of the microspheres, FITC and RITC, respectively, were combined with mussel adhesive protein fp-151, and they were observed by fluorescence microscopy and SEM. The results are shown in FIG. 7.

As shown in FIG. 7, it was confirmed that the diameter of the formed porous microsphere was about 220 μm, and the porous microsphere has a three-dimensional porous microsphere having pores connected to each other. Accordingly, it was confirmed that the porous microsphere can be used as a cell scaffold in which cells can attach and proliferate efficiently.

### Example 2. Analysis of Physical and Chemical Characterization of Mussel Adhesive Protein fp-151-Based Porous Microspheres

2-1. Analysis of FT-IR Spectroscopy of Porous Microsphere Surfaces

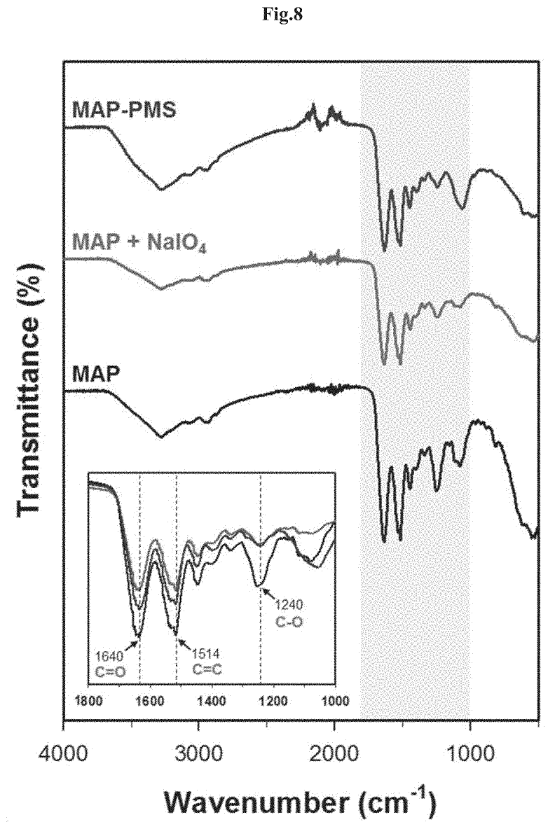

FT-IR spectroscopy analysis was performed to confirm the chemical bonding of the surface of the mussel adhesive protein-based porous microspheres (MAP-PMS) prepared in Example 1, and the results are shown in FIG. 8. As a comparison group, the modified fp-151 (MAP) and NaIO₄-treated fp-151 (MAP+NaIO₄) coated surfaces were used.

As shown in FIG. 8, it was confirmed that the porous microsphere had a peak similar to that of the fp-151 coated surface treated with NaIO₄ at a wavelength of about 1240 cm⁻¹ indicating C—O bonds in the catechol. As a result, it was confirmed that DOPA was changed into the DOPA-quinone by the oxidizing agent, allowing sufficient crosslinking progress to form microspheres.

2-2. Charge Measurement on Porous Microsphere Surfaces

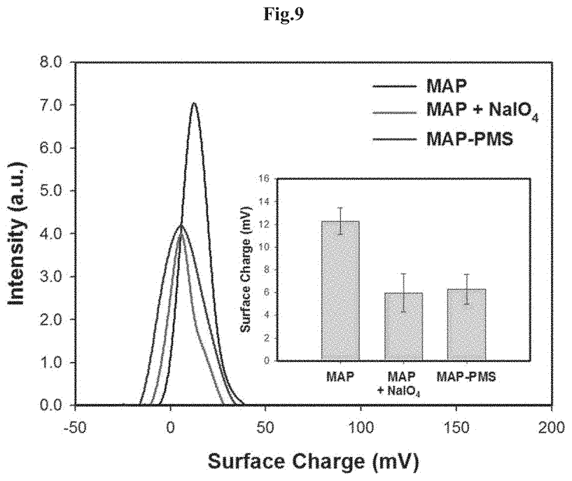

In order to determine the charge of the surface of the formed porous microsphere, the zeta potential was confirmed by introducing PBS of pH 7.4. As a comparison group, a solution in which the modified fp-151 was dissolved in PBS at a concentration of 1 mg/ml and a solution treated with NaIO₄ were used. The results are shown in FIG. 9.

As shown in FIG. 9, it was confirmed that the porous microsphere had a zeta potential (about +6 mV) similar to that of the fp-151 solution treated with NaIO₄.

2-3. Analysis of Pore Distribution on the Sectional Surface of Porous Microsphere

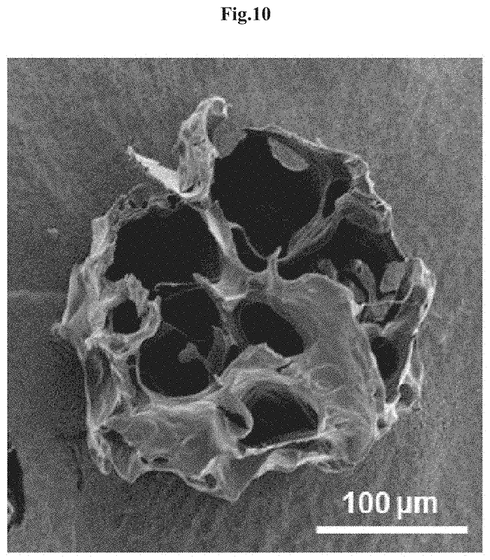

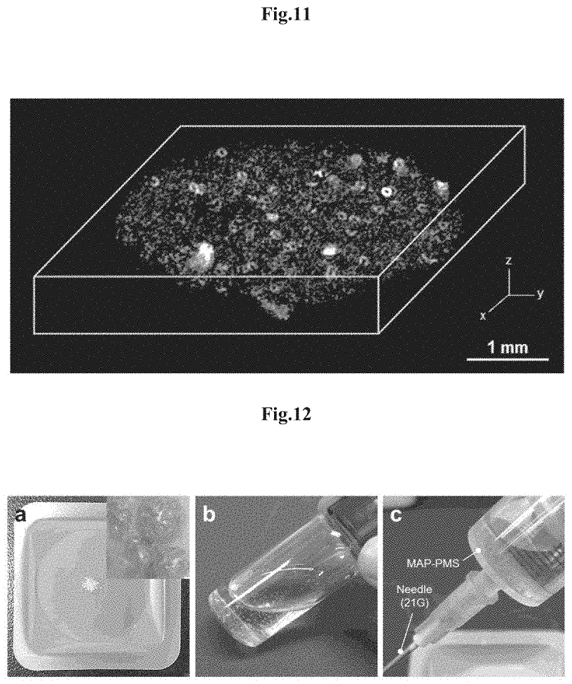

In order to closely examine the pore structure of the porous microsphere, the microspheres were cut and analyzed by SEM and micro-computed tomography (micro-CT). The results are shown in FIGS. 10 and 11.

As shown in FIGS. 10 and 11, it was confirmed that the pores having a diameter of 20 μm to 30 μm exist in the form connected to each other, and the porosity is about 95%.

### Example 3. Confirmation of Applicability of Mussel Adhesive Protein fp-15-Based Porous Microspheres

In order to confirm the applicability of the porous microspheres, the porous microspheres were introduced into the syringe, and the release pattern of the porous microspheres was observed. In addition, a mold was used to prepare the porous microspheres in a cylindrical structure. The adhesion was confirmed on the skin surface of the animal tissue with alginate microspheres. The results are shown in FIGS. 12 to 15.

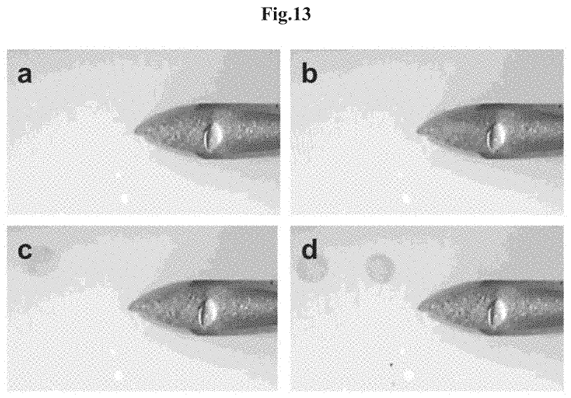

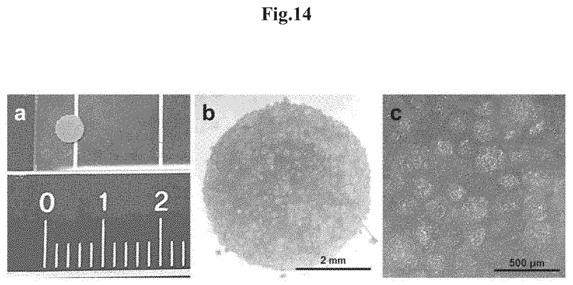

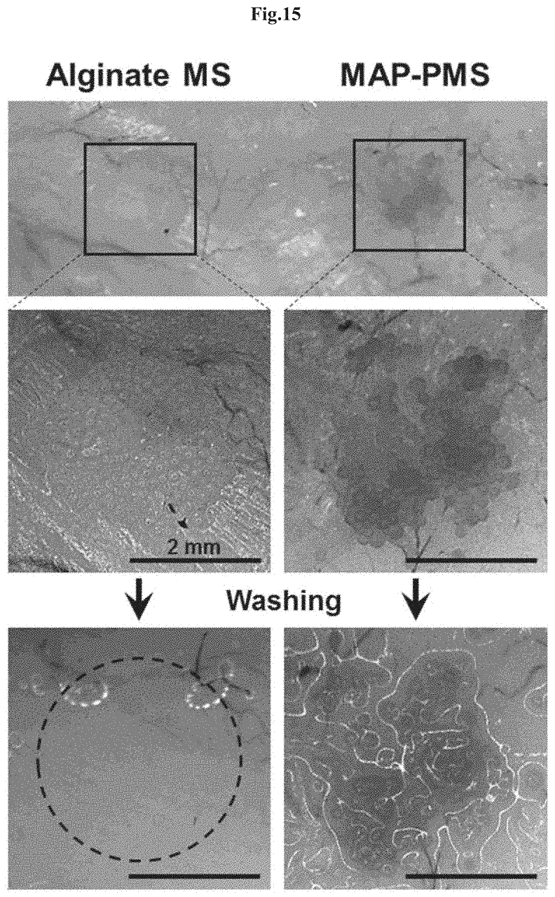

As shown in FIGS. 12 and 13, it was confirmed that the microspheres can be injected to the outside through the syringe while maintaining the form. In addition, as shown in FIG. 14, it was confirmed that the microspheres can be produced in a cylindrical structure by using a mold. Further, as shown in FIG. 15, when the microspheres are applied to the surface of the skin tissue of the animal through a syringe, it was confirmed that they are attached to the application site even after several times of wash due to their high adhesive property in the moist environment compared to the alginate microspheres control. This confirmed that the porous microspheres can be effectively applied to the site that requires tissue regeneration and recovery.

### Example 4. Analysis of in Vitro Cell Delivery Characterization of Mussel Adhesive Protein-Based Porous Microspheres

4-1. Confirmation of Cell Attachment Efficiency of Porous Microspheres

In order to confirm the efficacy of the mussel adhesive protein-based porous microsphere prepared in Example 1 as a cell carrier, mesenchymal stem cells (rMSCs), periodontal ligament stem cells (hPDLCs) and human umbilical vein endothelial cells (HUVECs) were used to perform In vitro cell experiments.

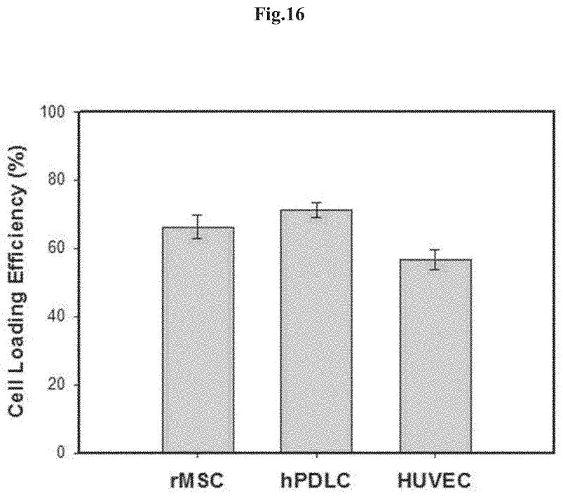

Specifically, 5×10⁴ cells per 1×10⁴ microspheres were mixed, and the mixture was incubated at 37° C. for 6 hours. The result of confirming the cell adhesion of each microsphere is shown in FIG. 16.

As shown in FIG. 16, it was confirmed that the cell loading efficiency with respect to the number of initially introduced cells is about 55% to about 70%.

4-2. Identification of Growth Patterns for Each Cell of the Porous Microsphere

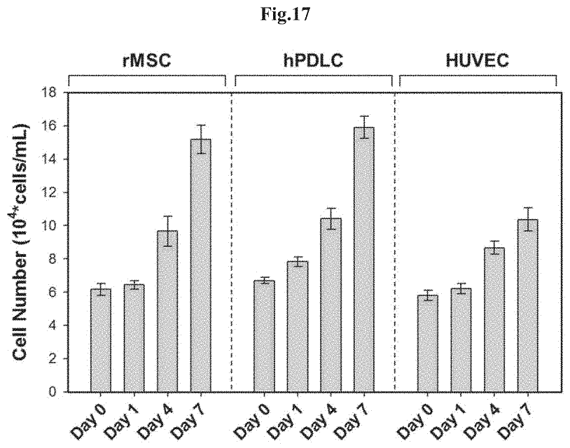

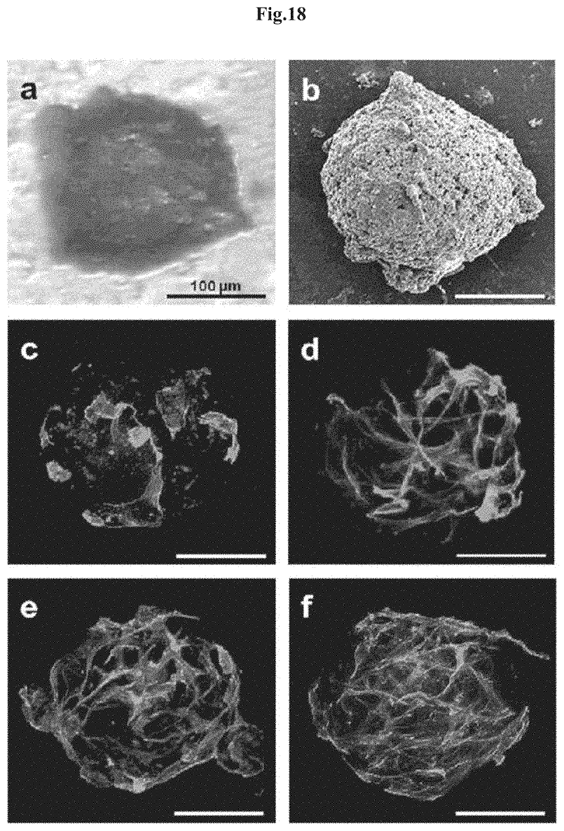

The growth pattern of each cell according to the culture date of the porous microsphere was confirmed. Specifically, the cells were cultured for 7 days with replacing the medium of each cell-microsphere complex every day. The results are shown in FIG. 17. In addition, in order to closely observe the attachment and growth of the mesenchymal stem cells to the porous microsphere, methylene blue staining was performed one day after attachment to the porous microsphere, and the optical microscope observation, SEM observation, and fluorescence analysis were performed. The results are shown in FIG. 18.

As shown in FIG. 17, efficient cell proliferation of all cells cultured in the porous microsphere was confirmed. Further, as shown in FIG. 18, the activity of the cells was maintained (FIG. 18A), and the cells were efficiently attached and proliferated on the surface and the pore surface of the porous microsphere (FIG. 18B). In addition, as a result of observing the actin fibers of the nucleus and cytoplasm through fluorescence staining, it was confirmed that as time lapses, the spreading pattern became active with the proliferation of cells (FIG. 18C).

### Example 5. Analysis of Characterization of Minimally Invasive in Vivo Bio-Injection of Mussel Adhesive Protein-Based Porous Microspheres

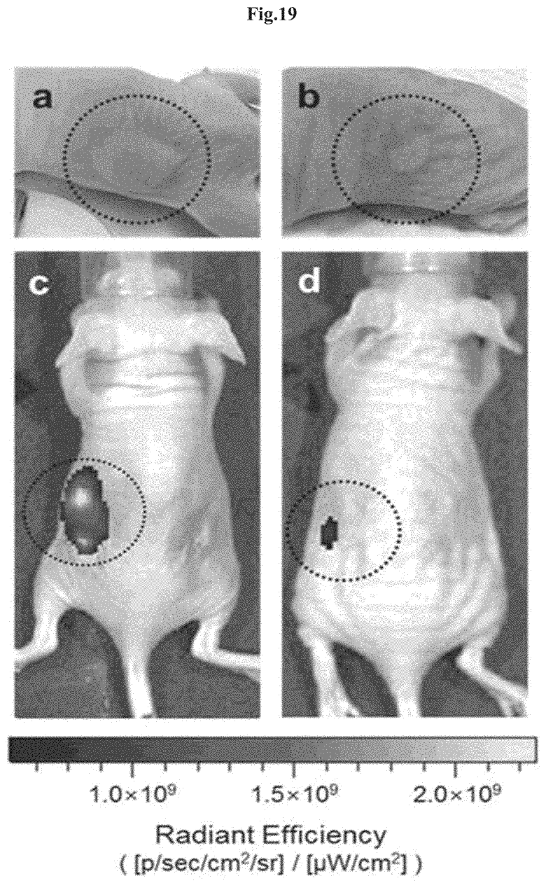

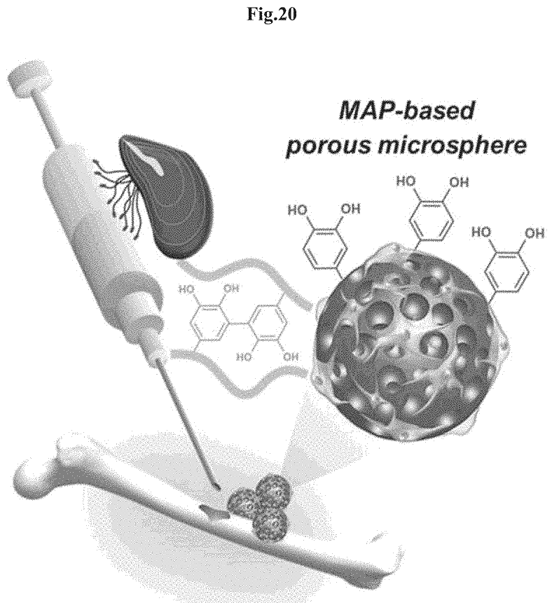

In order to confirm the minimally invasive bio-injection characteristics of the mussel adhesive protein-based porous microsphere prepared in Example 1, 1×10⁵ FITC-conjugated microspheres were mixed in PBS solution, and then the mixture was subcutaneously injected into 5 weeks old immunodeficient nude mouse (Balb/C Nude). On the day of injection and after 28 days of injection, the behavior of microspheres in vivo was confirmed using an in vivo fluorescence imaging device. The results are shown in FIG. 19.

As shown in FIG. 19, it was confirmed that the microspheres were successfully injected in a minimally invasive manner and remained to some extent even after 28 days.

Accordingly, it was confirmed that the mussel adhesive protein-based adhesive microspheres of the present invention can efficiently deliver therapeutic stem cells to tissue damage sites as cell carriers by minimally invasive bio-injection through syringes. In addition, it was confirmed that the present invention can be widely applied as a scaffold for tissue engineering or drug carrier that can be suitably applied to the size of the defected site of tissue.

1. A porous microsphere comprising a mussel adhesive protein.

2. The porous microsphere of claim 1, wherein the mussel adhesive protein is a protein consisting of an amino acid sequence selected from the group consisting of amino acids sequences represented by SEQ ID NO: 1, SEQ ID NO: 5, SEQ ID NO: 6, SEQ ID NO: 7, SEQ ID NO: 8 and SEQ ID NO: 9 or a fusion protein linked to one or more amino acid sequences selected from the group.

3. The porous microsphere of claim 2, wherein the fusion protein is the fusion protein comprising an amino acid sequence selected from the group consisting of amino acid sequences represented by SEQ ID NO: 10, SEQ ID NO: 12, SEQ ID NO: 13, SEQ ID NO: 14, and SEQ ID NO: 15.

4. The porous microsphere of claim 1, wherein 10% to 100% of total tyrosine residues of the mussel adhesive protein is modified to DOPA.

5. The porous microsphere of claim 1, wherein the porous microsphere is formed from a water-in-oil emulsion.

6. The porous microsphere of claim 5, wherein the water-in-oil emulsion is condensed through crosslinking by oxidation of DOPA residues contained in the mussel adhesive protein.

7. The porous microsphere of claim 1, wherein the porous microsphere has a porosity of 50% to 95%.

8. The porous microsphere of claim 1, wherein the porous microsphere has biocompatibility.

9. The porous microsphere of claim 1, wherein the porous microsphere is loaded or attached with a bioactive material.

10. The porous microsphere of claim 9, wherein the bioactive material is at least one bioactive material selected from the group consisting of a cell, a protein, a polypeptide, a polysaccharide, a monosaccharide, an oligosaccharide, a fatty acid, and a nucleic acid.

11. The porous microsphere of claim 10, wherein the cell is at least one cell selected from the group consisting of a mesenchymal stem cell, an adipocyte stem cell, an osteoblast, a periodontal ligament cell, a vascular endothelial cell, a fibroblast, a hepatocyte, a neuron, a cancer cell, B cell and a white blood cell.

12. The porous microsphere of claim 1, wherein the porous microsphere is minimally invasive bio-injectable.

13. A composition for forming a porous microsphere comprising a mussel adhesive protein.

14. The composition of claim 13, further comprising an oxidizing agent and a porogen.

15. The composition of claim 14, the oxidizing agent is one selected from the group consisting of NaIO₄, NaIO₃, VOSO₄, Na₃VO₄, Na₂Cr₂O₇, Mn(OAc)₃, MnO₂, KMnO₄, Na₂S₂O₈, H₂O₂, Na₂S₂O₄, BHP (tert-butyl hydroperoxide) and DTT (dithiothreitol).

16. A cell carrier comprising the porous microsphere of any one of claims 1 to 12.

17. A scaffold for tissue engineering, the scaffold comprising the porous microsphere of any one of claims 1 to 12.

18. A drug carrier comprising the porous microsphere of any one of claims 1 to 12.

19. A method for preparing a porous microsphere, the method comprising:
1) forming a water-in-oil emulsion comprising a mussel adhesive protein, an oxidizing agent and a porogen inside a microfluidic channel;
2) crosslinking the water-in-oil emulsion through oxidation of DOPA residues contained in the mussel adhesive protein; and
3) washing the emulsion particle obtained after crosslinking and forming pores.

20. A method for cell delivery, the method comprising administering the porous microsphere of claim 1 to a subject.

21. A method for drug delivery, the method comprising administering the porous microsphere of claim 1 to a subject.


SEQUENCE LISTING (25 sequences)

SEQ ID NO: 1 - protein, 800 aa, Artificial
  fp-1
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 2 - protein, 10 aa, Artificial
  fragment sequence derived from fp-1
AKPSYPPTYK

SEQ ID NO: 3 - protein, 120 aa, Artificial
  fp-1 variant
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 4 - protein, 126 aa, Artificial
  fp-1 variant-RGD
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKGRGDSP

SEQ ID NO: 5 - protein, 472 aa, Artificial
  fp-2
LFSFFLLLTCTQLCLGTNRPDYNDDEEDDYKPPVYKPSPSKYRPVNPCLKKPCKYNGVCKPRGGSYKCFCKGGYYGYNCNLKNACKPNQCKNKSRCVPVGKTFKCVCRNGNFGRLCEKNVCSPNPCKNNGKCSPLGKTGYKCTCSGGYTGPRCEVHACKPNPCKNKGRCFPDGKTGYKCRCVDGYSGPTCQENACKPNPCSNGGTCSADKFGDYSCECRPGYFGPECERYVCAPNPCKNGGICSSDGSGGYRCRCKGGYSGPTCKVNVCKPTPCKNSGRCVNKGSSYNCICKGGYSGPTCGENVCKPNPCQNRGRCYPDNSDDGFKCRCVGGYKGPTCEDKPNPCNTKPCKNGGKCNYNGKIYTCKCAYGWRGRHCTDKAYKPNPCVVSKPCKNRGKCIWNGKAYRCKCAYGYGGRHCTKKSYKKNPCASRPCKNRGKCTDKGNGYVCKCARGYSGRYCSLKSPPSYDDDEY

SEQ ID NO: 6 - protein, 50 aa, Artificial
  fp-3
PWADYYGPKYGPPRRYGGGNYNRYGRRYGGYKGWNNGWKRGRWGRKYYGS

SEQ ID NO: 7 - protein, 750 aa, Artificial
  fp-4
YGRRYGEPSGYANIGHRRYYERAISFHRHSHVHGHHLLHRHVHRHSVLHGHVHMHRVSHRIMHRHRVLHGHVHRHRVLHNHVHRHSVLHGHVHRHRVLHRHVHRHNVLHGHVHRHRVLHKHVHNHRVLHKHLHKHQVLHGHVHRHQVLHKHVHNHRVLHKHLHKHQVLHGHVHTHRVLHKHVHKHRVLHKHLHKHQVLHGHIHTHRVLHKHLHKHQVLHGHVHTHRVLHKHVHKHRVLHKHLHKHQVLHGHVHMHRVLHKHVHKHRVLHKHVHKHHVVHKHVHSHRVLHKHVHKHRVEHQHVHKHHVLHRHVHSHHVVHSHVHKHRVVHSHVHKHNVVHSHVHRHQILHRHVHRHQVVHRHVHRHLIAHRHIHSHQAAVHRHVHTHFEGNFNDDGTDVNLRIRHGIIYFGGNTYRLSGGRRRFMTLWQECLESYGDSDECFVQLLEGNQHLFTVVQGHHSTSFRSDLSNDLHPDNNIEQIANDHVNDIAQSTDGDINDFADTHYNDVAPIADVHVDNIAQTADNHVKNIAQTAHHHVNDVAQIADDHVNDIGQTAYDHVNNIGQTADDHVNDIAQTADDHVNAIAQTADDHVNAIAQTADDHVNDIGDTANSHIVRVQGVAKNHLYGINKAIGKHIQHLKDVSNRHIEKLNNHATKNLLQSALQHKQQTIEREIQHKRHLSEKEDINLQHENAMKSKVSYDGPVFNEKVSVVSNQGSYNEKVPVLSNGGGYNGKVSALSDQGSYNEGYAY

SEQ ID NO: 8 - protein, 82 aa, Artificial
  fp-5
KHHHHHHSSEEYKGGYYPGNTYHYHSGGSYHGSGYHGGYKGKYYGKAKKYYYKYKNSGKYKYLKKARKYHRKGYKKYYGGSS

SEQ ID NO: 9 - protein, 103 aa, Artificial
  fp-6
IAALCGIVKSIDSDDSDYDYKGRGYCTNKGCRSGYNYFGNKGYCKYGEKSYTYNCNSYAGCCLPRNPYGKLKYYCTNKYGCPNNYYFYNNKGYYYLEHHHHHH

SEQ ID NO: 10 - protein, 200 aa, Artificial
  fp-151
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGSSGSAKPSYPPTYKAKPSYPPTYKAKPSYPPTYKAKPSYPPTYKAKPSYPPTYKAKPSYPPTYKL

SEQ ID NO: 11 - protein, 202 aa, Artificial
  fp-151-RGD
MAKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGSSAKPSYPPTYKAKPSYPPTYKAKPSYPPTYKAKPSYPPTYKAKPSYPPTYKAKPSYPPTYKGRGDSP

SEQ ID NO: 12 - protein, 171 aa, Artificial
  fp-131
AKPSYPPTYKAKPSYPPTYKAKPSYPPTYKAKPSYPPTYKAKPSYPPTYKAKPSYPPTYKPWADYYGPKYGPPRRYGGGNYNRYGRRYGGYKGWNNGWKRGRWGRKYYGSAKPSYPPTYKAKPSYPPTYKAKPSYPPTYKAKPSYPPTYKAKPSYPPTYKAKPSYPPTYKL

SEQ ID NO: 13 - protein, 175 aa, Artificial
  fp-353
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 14 - protein, 187 aa, Artificial
  fp-153
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 15 - protein, 187 aa, Artificial
  fp-351
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSAKPSYPPTYKAKPSYPPTYKAKPSYPPTYKAKPSYPPTYKAKPSYPPTYKAKPSYPPTYK

SEQ ID NO: 16 - protein, 3 aa, Artificial
  RGD Group 1
RGD

SEQ ID NO: 17 - protein, 4 aa, Artificial
  RGD Group 2
RGDS

SEQ ID NO: 18 - protein, 4 aa, Artificial
  RGD Group 3
RGDC

SEQ ID NO: 19 - protein, 4 aa, Artificial
  RGD Group 4
RGDV

SEQ ID NO: 20 - protein, 10 aa, Artificial
  RGD Group 5
RGDSPASSKP

SEQ ID NO: 21 - protein, 5 aa, Artificial
  RGD Group 6
GRGDS

SEQ ID NO: 22 - protein, 6 aa, Artificial
  RGD Group 7
GRGDTP

SEQ ID NO: 23 - protein, 6 aa, Artificial
  RGD Group 8
GRGDSP

SEQ ID NO: 24 - protein, 7 aa, Artificial
  RGD Group 9
GRGDSPC

SEQ ID NO: 25 - protein, 5 aa, Artificial
  RGD Group 10
YRGDS